(19) United States
(12) Patent Application Publication
(10) Pub. No.: US 2008/0114286 A1
(43) Pub. Date: May 15, 2008

(54) Infusion Catheter

(76) Inventors: Kory P. Hamel, Bloomington, MN (US); Justin M. Crank, Maple Grove, MN (US)

Correspondence Address: AMS RESEARCH CORPORATION, 10700 BREN ROAD WEST, MINNETONKA, MN, 55343, US

(21) Appl. No.: 11/938,615

(22) Filed: Nov. 12, 2007

Related U.S. Application Data
(60) Provisional application No. 60/865,235, filed on Nov. 10, 2006.

(51) Int. Cl. A61N 1/30; A61M 25/10; A61N 7/00; A61M 27/00

(52) U.S. Cl. 604/21; 604/101.05; 604/22; 604/544

(57) ABSTRACT

Devices, methods and systems for delivering drugs and therapeutic fluids to the prostate without requiring piercing of the prostate. An infusion catheter includes at least two inflatable balloons including a distal balloon providing a seal between the bladder and the prostatic urethra as well as a proximal balloon for sealing off the prostatic urethra from the penal urethra. The infusion catheter can include a number of lumens such as, for example, a drug delivery lumen, a drainage lumen, and one or more inflation lumens. The infusion catheter can have one or more drug delivery ports located between the distal balloon and the proximal balloon, wherein each drug delivery port is in fluid communication with the drug delivery lumen. In addition, the catheter can include one or more intermediate balloons at selected locations between the proximal balloon and the distal balloon for isolating portions of the prostate from exposure to treatment.

### PRIORITY CLAIM

The present application claims priority to U.S. Provisional Application Ser. No. 60/865,235, filed Nov. 10, 2006 and entitled, “INFUSION CATHETER”, which is hereby incorporated by reference in its entirety.

### FIELD OF THE INVENTION

The present invention relates generally to treatments of the prostate. More specifically, the present invention is a drug delivery catheter having inflatable balloons to isolate the prostatic urethra for delivery of a drug therapy.

### BACKGROUND OF THE INVENTION

Prostate health is of great concern for males and risks generally increase with age. While prostate cancer (prostatic carcinoma) receives many of the headlines as a leading cause of death for males, a variety of other afflictions can affect the prostate including prostatitis and benign prostatic hyperplasia (BPH, also known as benign prostatic hypertrophy).

Prostatitis is an inflammation of the prostate gland. Symptoms of prostatitis can include difficult urination, burning or painful urination, perineal or lower back pain, joint or muscle pain, tender or swollen prostate, blood in the urine, or painful ejaculation. Prostatitis is caused by bacterial infection in many instances, in which case treatment generally includes antimicrobial medication. Noninfectious forms of prostatitis are treated by other means, such as administration of an α₁-adrenoreceptor antagonist drug to relax the muscle tissue in the prostate and reduce the difficulty in urination.

Benign prostatic hypertrophy is a very common disorder, affecting an estimated 12 million men in the United States alone. BPH is a chronic condition and is strongly age-related. It is estimated that approximately 50% of men over the age of fifty, 75% of men beyond the age of seventy, and 90% of men over the age of eighty are afflicted with BPH. BPH is a non-cancerous condition characterized by enlargement of the prostate, obstruction of the urethra and gradual loss of bladder function. Representative symptoms can include difficult urination, frequent urination, incomplete emptying of the bladder, and urgency of urination.

BPH can be treated with a number of therapeutic modalities including surgical and medicinal methods. Transurethral resection of the prostate (TURP) is one preferred surgical method of treating BPH. A typical TURP procedure requires general anesthesia, and the placement of a resectoscope in the urethra for removal of multiple small chips of hyperplastic prostatic tissue, to relieve the obstruction. Complications from TURP can include bleeding, incontinence, retrograde ejaculation and impotence.

An alternative surgical method for treating BPH is transurethral incision of the prostate (TUIP). In the TUIP procedure, incisions are made in the prostate to relieve pressure and improve flow rate. Incisions are made where the prostate meets the bladder. No tissue is removed in the TUIP procedure. Cutting muscle in this area relaxes the opening to the bladder, which decreases resistance to urine flow from the bladder. A variant of the TUIP procedure in which a laser is used to make the incision is known as transurethral laser incision of the prostate (TULIP).

Other surgical methods used to relieve the symptoms of BPH include methods of promoting necrosis of tissue that blocks the urethra. Hyperthermic methods, for example, use the application of heat to “cook” tissue and kill the cells. The necrosed tissue is gradually absorbed by the body. Several methods of applying heat or causing necrosis have been utilized, including direct heat (transurethral needle ablation, or TUNA), microwave (transurethral microwave treatment, or TUMT), ultrasound (high-intensity focused ultrasound, or HIFU), electrical vaporization (transurethral electrical vaporization of the prostate, or TUEVP) and laser ablation (visual laser ablation of the prostate, or VLAP), among others.

Chemical ablation (chemoablation) techniques for promoting prostate tissue necrosis are also currently under development. In one chemical ablation technique, absolute ethanol is injected transurethrally into the prostate tissue. This technique is known as transurethral ethanol ablation of the prostate (TEAP). The injected ethanol causes cells of the prostate to burst, killing the cells. The prostate shrinks as the necrosed cells are absorbed. Generally no tissue sloughing is observed with this technique. As a treatment for BPH transurethral injection of ethanol is cost-effective and is reported to have few complications; see Goya, et al., J. Urol. 162, 383 (1999). Transperineal ethanol injection has been reported to be effective in the treatment of BPH; see Savoca, et al., Eur. Urol. 40, 504 (2001). Ethanol ablation methods have also been investigated for treatment of prostatic carcinoma (Amano, et al., Urology 59, 771 (2002)) and liver cancer (Livraghi, et al., Cancer 83, 48 (1998)).

Several drugs have been approved in the United States for the treatment of BPH. One class of drugs used in treating BPH is the inhibitors of the enzyme 5α-reductase. Also approved for treatment of BPH are the α₁-adrenoreceptor antagonist (or α-blocker) drugs, including terazosin (HYTRIN, doxazosin (CARDURA) and tamsulosin FLOMAX). For additional discussion of drug treatment of BPH, reference is made to U.S. Pat. No. 7,015,253 to Escandon et al., the disclosure of which is hereby incorporated by reference in its entirety.

Prostate cancer is a common cancer among males and a leading cause of cancer deaths in males beyond the age of fifty. Prostate cancer generally begins as a tumor on the prostate gland. Prostate cancer that has been confined to the gland can often be treated successfully. If left untreated, the cancer may spread to surrounding tissues near the prostate, to seminal vesicles, and to distant parts of the body, such as bones, liver, or lungs. Often, prostate cancer is slow-growing permitting a course of treatment referred to as “watchful waiting” for elderly patients or for patients in otherwise poor health. More aggressive treatments are required if the disease progresses. A variety of treatments exist for prostate cancer and can include radiation, cryosurgery, brachytherapy, chemical treatment, surgery, and drug therapy (including antiandrogen drugs, hormones, and steroids).

In conducting certain prostate therapies, the prostate and surrounding tissues are pierced, such as, for example, with drug injection. However, there are a variety of undesirable side effects related to piercing the prostate and surrounding tissues including pain, inflammation, and infection. A need exists, therefore, for a prostate treatment that does not require piercing the prostate or surrounding tissue.

### SUMMARY OF THE INVENTION

The present invention generally relates to devices and methods for delivering drugs and therapeutic fluids to the prostate, or other selected urogenital tract organs, without requiring the organ be pierced to administer the treatment. A catheter is provided having at least two inflatable balloons including a proximal balloon and a distal balloon. The distal balloon is inflated within the bladder, providing a seal between the bladder and the prostatic urethra. The proximal balloon is inflated within the penile portion of the urethra, thereby sealing off the prostatic urethra. Within the catheter, a number of lumens can be provided including, for example, a drug delivery lumen, a drainage lumen, and one or more inflation lumens. The catheter can include one or more drug delivery ports located between the distal balloon and the proximal balloon, wherein each drug delivery port is in fluid communication with the drug delivery lumen. The catheter can further comprise one or more intermediate balloons provided on the catheter at selected locations between the proximal balloon and the distal balloon.

In one aspect of the present invention, an infusion catheter can comprise at least two inflatable balloons including a proximal balloon and a distal balloon. The catheter can further comprise one or more intermediate balloons oriented on the catheter at positions between the proximal balloon and the distal balloon for selectively sealing treatment portions of the prostate from exposure to a drug or therapeutic fluid. Preferably, the one or more intermediate balloons are located within the prostatic urethra, and may be concentrically or non-concentrically disposed on the catheter. For instance, the one or more intermediate balloons can be used to seal off and isolate prostatic or ejaculatory ducts. The catheter can contain a variety of isolated lumens including, for example, a drug delivery lumen, a drainage lumen, and one or more inflation lumens. The catheter can further include one or more drug delivery ports located between the distal balloon and the proximal balloon for delivering a drug or therapeutic fluid to selected treatment locations with the prostate. The drug or other therapeutic fluid to be administered is introduced into the catheter with the drug delivery lumen. The drug delivery source may or may not be administered under pressure. Further, the drug may be delivered with a single administration or may be delivered using a plurality of individual pulses.

In another aspect of the present invention, a method for delivering a drug or therapeutic fluid to the prostate can include positioning an infusion catheter within the urethra such that a distal balloon can be inflated to isolate the prostate urethra from the bladder while a proximal balloon is inflated to isolate the prostate urethra from a penile portion of the urethra to define a treatment portion. Once the prostate urethra has been isolated, a drug or other therapeutic fluid can be administered to a desired treatment site within the prostate through one or more drug delivery ports in the catheter. In some embodiments, one or more intermediate balloons can be inflated within the prostate urethra to isolate portions of the prostate from exposure to treatment such as, for example, the prostatic or ejaculatory ducts. In some embodiments, the drug or therapeutic fluid can be delivered with a single administration or alternatively can be administered with a plurality of individual pulses.

In another aspect of the present invention, a prostate treatment system can comprise an infusion catheter and a pressurized drug delivery source. The infusion catheter can comprise at least a distal balloon for isolating the prostate urethra from the bladder and a proximal balloon for isolating the prostate urethra from a penal portion of the urethra. In some embodiments, the infusion catheter can further comprise one or more intermediate balloons for inflation within the prostate urethra so as to isolate selected portions of the prostate from exposure to treatment. The pressurized drug delivery source can supply a drug or other therapeutic fluid to the prostate through one or more drug delivery ports in the infusion catheter.

The above summary of the various representative embodiments of the invention is not intended to describe each illustrated embodiment or every implementation of the invention. Rather, the embodiments are chosen and described so that others skilled in the art may appreciate and understand the principles and practices of the invention. The figures in the detailed description that follows more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is an anatomical drawing depicting the location of the prostate.

FIG. 2 is an anatomical drawing featuring an infusion catheter according to one embodiment of the present invention.

FIG. 3 is an anatomical drawing featuring an infusion catheter according to a further embodiment of the present invention.

FIG. 4 is a cross sectional view of the infusion catheter of the present invention.

### DETAILED DESCRIPTION OF THE DRAWINGS

In the following detailed description of the present invention, numerous specific details are set forth in order to provide a thorough understanding of the present invention. However, it will be obvious to one skilled in the art that the present invention may be practiced without these specific details. In other instances, well-known methods, procedures, and components have not been described in detail so as to not unnecessarily obscure aspects of the present invention.

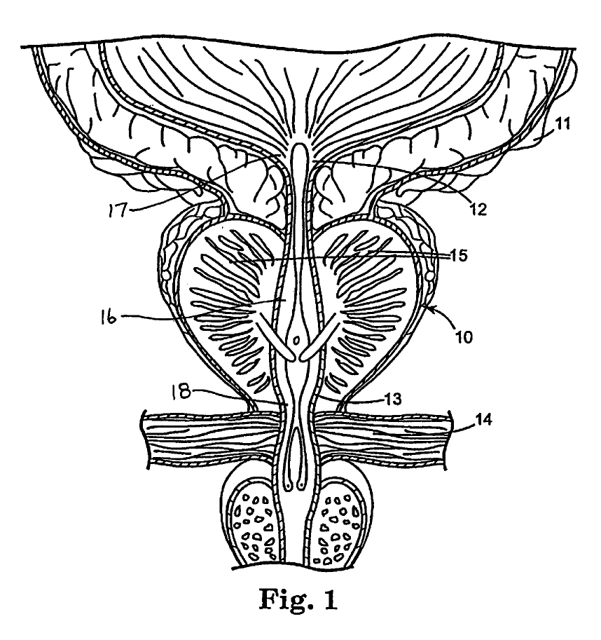

Referring now to FIG. 1, the anatomical position of prostate 10 is depicted, with prostate 10 (including lateral lobes 15) surrounding urethra 13, and adjacent tissue including seminal vesicles 11, bladder neck 12, and pelvic tissues including sphincter muscles 14. Prostatic urethra 16 includes a bladder end 17 and a penile end 18.

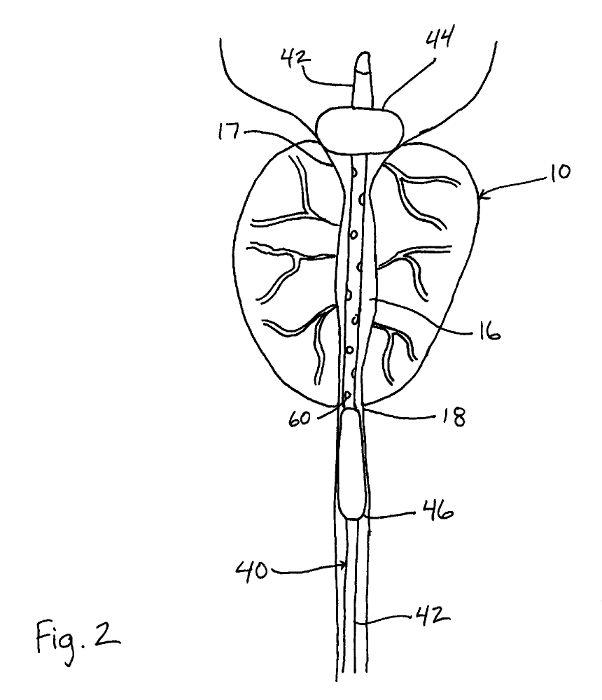

As illustrated in FIGS. 2-4, an infusion system 40 is depicted, comprising an infusion catheter 42, a distal balloon 44 and a proximal balloon 46. Infusion catheter 42 can substantially resemble a Foley catheter and can comprise multiple individual lumens contained within the infusion catheter 42. The infusion catheter 42 can include a drug delivery lumen 52 in fluid communication with one or more delivery ports 60 disposed on infusion catheter 42 between the distal balloon 44 and the proximal balloon 46. In addition, infusion catheter 42 can include a drainage lumen 50 adapted to drain urine from the bladder as well as one or more inflation lumens 54 adapted to inflate one or more of the balloons of infusion system 40.

Distal balloon 44 is coupled to infusion catheter 42 and is adapted to be inflated or deflated through the use of an inflation lumen 54 within the infusion catheter 42. Distal balloon 44 is coupled to the distal end of infusion catheter 42 and is positioned and inflated within a patient's bladder so as to seal bladder end 17 of prostatic urethra 16. Distal balloon 44 is situated generally at bladder neck 12. The use of inflatable balloons with catheters in various medical treatments is known, for example in cardiovascular treatments and urinary treatments. A discussion of inflatable balloon catheters is included within U.S. Pat. No. 6,312,405 to Meyer et al., the disclosure of which is incorporated herein by reference in its entirety.

Proximal balloon 46 is coupled to catheter 42, and is adapted to be inflated or deflated with the use of an inflation lumen within the infusion catheter 42. Proximal balloon 46 is coupled to catheter 42 such that it can be inflated within the penile portion of a patient's urethra. When inflated, proximal balloon 46 seals penile end 18 of prostatic urethra 16.

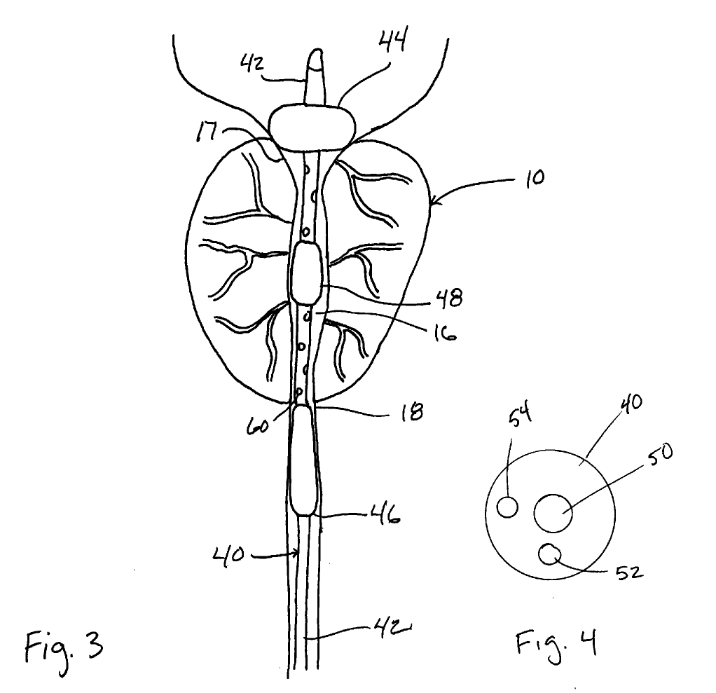

In one representative embodiment of the infusion system 40 as illustrated in FIG. 3, infusion device 40 can include one or more intermediate balloons 48 coupled to infusion catheter 42. Intermediate balloons 48 are generally positioned at selected locations between the distal balloon 44 and proximal balloon 46. The intermediate balloon 48 is arranged to be inflated within prostatic urethra 16, and is configured to seal off or block portions of prostatic urethra 16 that are not to be infused with the drug treatment such as the prostatic or ejaculatory ducts. The one or more intermediate balloons 48 can be arranged concentrically or non-concentrically about the infusion catheter 42 based upon the structure to be isolated with the intermediate balloons.

The various balloons of infusion device 40 can be arranged on infusion catheter 42 so as to share a common inflation lumen 54, or alternatively, each balloon may be operably coupled to its own inflation lumen 54. In addition, the inflation pressure of each balloon may be varied in accordance with the desired sealing application.

Referring to FIGS. 2 and 3, infusion catheter 42 can further comprise one or more delivery ports 60. Each delivery port 60 is in fluid communication with a delivery lumen 52 within the infusion catheter 42 for delivery a drug or therapeutic fluid from a pressurized drug delivery source.

In use, infusion device 40 is oriented such that infusion catheter 42 can be inserted into a patient's urethra following well known procedures similar to inserting a Foley-type urological catheter. During insertion of the infusion catheter 42, each of the balloons is in a deflated state so as to facilitate the insertion of infusion catheter 42. Infusion catheter 42 is positioned in the approximate desired location, such that deflated distal balloon 44 is within the patient's bladder. Distal balloon 44 can then be inflated through an inflation lumen 54 within the infusion catheter 42, and a liquid or gas may be used to inflate the distal balloon 44. As distal balloon 44 is inflated, the position of catheter 42 may be adjusted in order to effectively seat distal balloon 44 against bladder end 17 of prostatic urethra 16 and provide a seal.

Proximal balloon 46 may be inflated separately or simultaneously with distal balloon 44. Similarly, if one or more intermediate balloons 48 is included with infusion catheter 42, they can be inflated separately or simultaneously with the other balloons. Proximal balloon 46 is inflated in the penile portion of a patient's urethra, and seated against penile end 18 of prostatic urethra 16. After inflation of distal balloon 44 and proximal balloon 46, prostatic urethra 16 is effectively sealed.

As described previously, one or more intermediate balloons 48 may optionally be used with infusion device 40. The location of intermediate balloons 48 on infusion catheter 42 is chosen based on the desired application. For example, it may be desired to block off a patient's ejaculatory ducts with intermediate balloon 48 so as to prevent exposure of the ejaculatory duct to a drug or therapeutic fluid.

To deliver a drug infusion therapy according to the present invention, no incision or puncture of the prostate or surrounding tissue is needed. Prostate 10 generally includes many ducts that are in communication with prostatic urethra 16, and perfusion of a drug of other therapeutic fluid into the ducts is possible, thereby administering a therapeutic agent throughout the prostate without puncturing any tissue.

A drug delivery lumen 52 within infusion catheter 42 is provided to introduce the chosen treatment. The drug or therapeutic fluid to be introduced may be pushed into infusion catheter 42 with a manual or automated pressure drug delivery source or alternatively, the drug or therapeutic fluid can be introduced with a vacuum. In some instances, a single administration of the drug or therapeutic fluid may prove beneficial while in other instances, the use of a pulsed, or cycled, delivery can prove beneficial. The drug or therapeutic fluid generally travels from a pressurized fluid source, through the infusion catheter 42 and out of one or more of the delivery ports 60. As the drug or therapeutic fluid exits the delivery ports 60, the drug or therapeutic fluid can flow into the prostatic urethra 16, wherein it can diffuse into the prostate through the urethral wall, or through the prostate ducts. Pressure may be applied to the drug delivery lumen 52 to increase the delivery of the drug or therapeutic fluid. As the prostatic urethra 16 is sealed off by the distal balloon 44 and the proximal balloon 46, the treatment introduced from delivery ports 60 is effectively transmitted to the afflicted prostate 10 without risking potential damage to healthy tissue.

In one embodiment, electroporation may be used in conjunction with the drug delivery method detailed herein, to increase the absorption of the drug or therapeutic fluid into prostate 10. In such an embodiment, electrodes may be provided on infusion device 40, or be provided on a separate device used in conjunction with infusion device 40. In a further embodiment, ultrasound may be used with the drug delivery method detailed herein, to increase absorption of the drug or therapeutic fluid into prostate 10. Methods and techniques relating to electroporation and ultrasound are generally well known to one of skill in the art.

Following treatment, infusion device 40 is removed by deflating the distal balloon 44, proximal balloon 46 and any intermediate balloons 48. Following deflation of the balloons, the infusion catheter 42 can be removed from the patient's urethra.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives.

1. An infusion catheter system for delivering a drug or therapeutic fluid during prostate treatment, said infusion catheter system including:
a first inflatable balloon;
a second inflatable balloon, wherein upon inflation said first and second inflatable balloons define a sealed portion of prostatic urethra;
a drug delivery portion, wherein said drug delivery portion is presented intermediate the sealed portion of prostatic urethra,
a drug delivery source operably connected to the drug delivery portion, wherein said drug delivery source provides a drug or therapeutic agent to said drug delivery portion and said drug delivery portion subsequently delivers the drug or therapeutic agent to the sealed portion of the prostatic urethra.

2. The infusion catheter system of claim 1 further including a third inflatable balloon intermediate said first and second inflatable balloons.

3. The infusion catheter system of claim 2 wherein the third balloon is non-concentrically located from said first and second inflatable balloons.

4. The infusion catheter system of claim 2, wherein the third balloon is concentrically located relative said first and second inflatable balloons.

5. The infusion catheter system of claim 2 further including a fourth inflatable balloon intermediate said first and second inflatable balloons.

6. The infusion catheter system of claim 1 further including a drainage lumen, wherein said drainage lumen drains urine that is blocked from exit from the bladder by said first and second inflatable balloons.

7. The infusion catheter system of claim 1 further including an inflation lumen to inflate the first balloon and the second inflatable balloons.

8. The infusion catheter system of claim 1 wherein the drug delivery source provides a pressurized drug or therapeutic agent to said drug delivery portion.

9. The infusion catheter system of claim 1 further including an electroporation system disposed proximate the drug delivery portion.

10. The infusion catheter system of claim 1 further including an ultrasound system disposed proximate the drug delivery portion.

11. The infusion catheter system of claim 1 wherein the drug delivery portion includes a plurality of delivery ports that are disposed concentrically about the drug delivery portion.

12. A method for delivering a drug or therapeutic fluid during prostate treatment, including the steps:
defining and sealing a first end of a prostatic urethra;
defining and sealing a second end of a prostatic urethra;
delivering a drug or therapeutic fluid between the sealed first and second end s through the urethra to the prostate gland.

13. A method for delivering a drug or therapeutic fluid during prostate treatment as described in claim 12 further including defining and sealing an intermediate portion of said prostatic urethra, between said first and second ends.

14. A method for delivering a drug or therapeutic fluid during prostate treatment as described in claim 12 further including multiple deliveries of the drug or therapeutic agent.

15. A method for delivering a drug or therapeutic fluid during prostate treatment as described in claim 12 further wherein the step of delivering a drug or therapeutic fluid comprises delivering a pressurized drug or therapeutic fluid.

16. A method for delivering a drug or therapeutic fluid during prostate treatment as described in claim 12 further including the step of electroporating the prostatic urethra.

17. A method for delivering a drug or therapeutic fluid during prostate treatment as described in claim 12 further including the step of applying ultrasound to the prostatic urethra.

18. An infusion catheter system for delivering a drug or therapeutic fluid to the prostate gland, said infusion catheter comprising:
prostatic urethra sealing means, including first sealing means and second sealing means, wherein said first sealing means for sealing off and defining a first end of a prostatic urethra and wherein said second sealing means for sealing off and defining a second end of a prostatic urethra; and
drug delivery means, wherein said drug delivery means defined between said first end and said second end of said prostatic urethra, wherein said drug delivery means for delivering a drug through the prostatic urethra to the prostate gland.

19. The infusion catheter system of claim 18, further comprising a bladder drainage means for draining urine from a patient's bladder that is blocked from exit by said prostatic urethra sealing means.

20. The infusion catheter system of claim 18, wherein said drug delivery means includes pressurization means for delivering said drug under pressure.
